(19) United States
(12) Patent Application Publication
(10) Pub. No.: US 2025/0391497 A1
(43) Pub. Date: Dec. 25, 2025

(54) System and method for transmission timeline generation

(71) Applicant: Solu Healthcare Oy, Helsinki (FI)

(72) Inventors: Timo Moilanen, Helsinki (FI); Kerkko Visuri, Helsinki (FI)

(21) Appl. No.: 18/748,217

(22) Filed: Jun. 20, 2024

(51) Int. Cl. G16B 10/00; G16B 20/20

(52) U.S. Cl. CPC G16B 10/00; G16B 20/20

(57) ABSTRACT

According to an example aspect of the present invention, there is provided a method for generating a transmission timeline, the method comprising: determining, based on SNP information, SNP evolutionary distance from a reference genome for each sample; determining, based on the SNP information, SNP evolutionary distance between each sample; and generating, based on: the SNP information, the SNP evolutionary distances from the reference genome, the SNP evolutionary distance between the samples, the mutation rate, generation rules and the corresponding timestamps; a dated phylogenetic tree, said tree comprising sample nodes) and non-sample nodes, wherein each sample may correspond to a node, for example.

### FIELD

At least some embodiments of the present disclosure relate to analysis and modelling of transmission dynamics of pathogenic microbes.

### BACKGROUND

Infectious diseases cause a significant risk to public health. Such risks are evident, for example, in the health care sector, for example in hospitals wherein a person, such as a patient with lowered immune defence may be in contact with, and infected by, a pathogenic microbe. Fast and reliable solutions for analysis and modelling of transmission dynamics needed to inhibit spread of infectious diseases, for example to inhibit antibiotic resistant strains of bacteria in a hospital environment.

### SUMMARY OF THE INVENTION

It is an aim of the present disclosure to generate a model relating to the transmission dynamics of a species, for example, a species of pathogenic microbes. A conventional phylogenetic tree does not typically provide information on temporal connections between samples or evolutionary progression of a species. The embodiments herein overcome these limitations by using computational methods and apparatuses in order to construct a transmission timeline, e.g. in the form of an updated dated phylogenetic tree.

The invention is defined by the features of the independent claims. Some specific embodiments are defined in the dependent claims.

According to a first aspect of the present invention, there is provided a method for generating a transmission timeline, the method comprising: obtaining a reference genome for a species; obtaining a timestamp for each sample of a plurality of samples, wherein at least one sample is from said species; obtaining sample genomic data corresponding to each sample; obtaining, based on a difference between the reference genome and each sample genomic data, SNP information from each sample in the plurality of samples; obtaining a mutation rate for said species; determining, based on the SNP information, SNP evolutionary distance from the reference genome for each sample; determining, based on the SNP information, SNP evolutionary distance between each sample; and generating, based on the SNP information, the SNP evolutionary distances from the reference genome, the SNP evolutionary distance between the samples, the mutation rate, generation rules and the corresponding timestamps, a dated phylogenetic tree, said tree comprising sample nodes) and non-sample nodes, wherein each sample may correspond to a node, for example.

According to a second aspect of the present invention, there is provided a method for generating a transmission timeline, the method comprising: obtaining a reference genome for a species; obtaining a timestamp for each sample of a plurality of samples, wherein at least one sample is from said species; receiving sequence reads of each sample from the species, and for sequence reads of each sample: assembling said sequence reads thereby obtaining sample genomic data corresponding to said sample; obtaining, based on a difference between the reference genome and each sample genomic data, SNP information from each sample in the plurality of samples; obtaining a mutation rate for said species; determining, based on the SNP information, SNP evolutionary distance from the reference genome for each sample; determining, based on the SNP information, SNP evolutionary distance between each sample; generating, based on the SNP information, the SNP evolutionary distances from the reference genome, the SNP distance between the samples, the mutation rate, generation rules and the corresponding timestamps, a dated phylogenetic tree, said tree comprising sample nodes and non-sample nodes, and wherein each sample corresponds to a node.

According to a third aspect of the present invention, there is provided a method for generating a transmission timeline, for example in relation to a pathogen, the method comprising: obtaining at least one sample from a sample source, wherein the sample source is, for example a patient, a healthcare worker, a medical device, an implement, or an environmental sample, wherein said sample comprises or is suspected to comprise at least one a pathogenic microbe; preparing a pure culture from said sample; isolating genomic DNA from said pure culture; sequencing the isolated genomic DNA to obtain sequence reads; obtaining a reference genome for a pathogenic microbe; assembling said sequence reads thereby obtaining sample genomic data; comparing SNPs of the sample genomic data to the reference genome, obtaining a timestamp for each sample of a plurality of samples, wherein at least one sample is from said pathogenic microbe; obtaining, based on a difference between the reference genome and each sample genomic data, SNP information from each sample in the plurality of samples; obtaining a mutation rate for said pathogenic microbe; determining, based on the SNP information, SNP evolutionary distance from the reference genome for each sample; determining, based on the SNP information, SNP evolutionary distance between each sample; generating, based on the SNP information, the SNP evolutionary distances from the reference genome, the SNP distance between the samples, the mutation rate, generation rules and the corresponding timestamps, a dated phylogenetic tree, said tree comprising sample nodes and non-sample nodes, wherein each sample corresponds to a node.

According to a fourth aspect of the present invention, there is provided a method for generating a transmission timeline, a) providing a sample from a patient or an environmental sample, wherein said sample is known or suspected to comprise a pathogenic microbe; b) preparing a pure culture from said sample; c) isolating genomic DNA from said pure culture; d) sequencing the genomic DNA obtained in step c); e) comparing the genomic sequencing data obtained from step d) to one or more reference sequences in order to identify and locate the presence or absence of SNPs in said genomic sequencing data; f) preparing SNP information, for example a SNP map, based on the SNPs identified and located in step e), wherein said SNP map comprises timestamp information and location information of the sample; g) preparing a dated phylogenetic tree by combining the SNP information with previous SNP information obtained from other samples and/or from a reference genome, said dated phylogenetic tree comprising sample nodes and non-sample nodes, and wherein each sample corresponds to a node.

According to a fifth aspect of the present invention, there is provided a non-transitory computer readable medium having stored thereon a set of computer readable instructions that, when executed by at least one processor, cause an apparatus to at least participate in performing any of the other aspects of the invention.

According to a sixth aspect of the present invention, there is provided a computer program product that, when executed by at least one processor, causes an apparatus to at least participate in performing any of the other aspects of the invention.

According to a seventh aspect of the present invention, there is provided an apparatus, comprising at least one processing core, at least one memory including computer program code, the at least one memory and the computer program code being configured to, with the at least one processing core, cause the apparatus at least to participate in performing any of the other aspects of the invention.

### BRIEF DESCRIPTION OF THE DRAWINGS

FIGS. 1A & 1B illustrate example apparatus capable of supporting at least some embodiments of the present disclosure;

FIG. 2A illustrates an example of a phylogenetic tree;

FIG. 2B illustrates an example of a dated phylogenetic tree;

FIG. 3 illustrates an example tree structure;

FIG. 4A illustrates an example dated phylogenetic tree;

FIG. 4B illustrates an example updated phylogenetic tree;

FIGS. 5A & 5B illustrate an example dated phylogenetic tree capable of supporting at least some embodiments of the present disclosure;

FIG. 6A illustrates example locations for sample source in accordance with at least some embodiments of the present disclosure;

FIGS. 6B & 6C illustrate an example method capable of supporting at least some embodiments of the present disclosure;

FIG. 6D illustrates SNP calling;

FIG. 7 illustrates an apparatus capable of supporting at least some embodiments of the present disclosure;

FIG. 8 illustrates a method capable of supporting at least some embodiments of the present disclosure; and

FIG. 9 illustrates a method capable of supporting at least some embodiments of the present disclosure.

### EMBODIMENTS

The term “genomic” refers to information obtainable from a genome or parts thereof. In other words, information may be obtained from coding regions (i.e., genes), and/or from non-coding regions of a genome. Such information may be, for example, single nucleotide polymorphisms, SNPs. SNP refers to a germline substitution of a single nucleotide at a specific position in the genome. In the context of the present disclosure, SNP is to be understood as a nucleotide substitution irrespective of its frequency or abundancy within a population. Thus, for example, terms such as “single nucleotide variant”, SNV, and “single nucleotide mutation” is to be understood to correspond to SNPs in the context of the present disclosure. Moreover, SNP may be seen as a relative concept compared to a suitable reference, such as a reference genome.

A “species” is to be understood as a collection of microbe strains, such as bacterial and/or fungal strains, that are genetically similar to a degree that distinguishes said strains from other groups of microbes. Said microbes may be pathogenic microbes. In at least some embodiments, the species is Escherichia coli. In at least some embodiments, the species is at least one of: Acinetobacter baumannii, Enterococcus faecium, Candida auris, Escherichia coli, Staphylococcus aureus, Pseudomonas aeruginosa, Klebsiella pneumoniae or Salmonella enterica. In at least some embodiments, the species is at least one of: Acinetobacter baumannii, Acinetobacter pittii, Candida auris, Enterobacter cloacae, Escherichia coli, Enterococcus faecium, Enterobacter hormaechei, Klebsiella pneumoniae, Klebsiella quasipneumoniae, Mycobacteroides abscessus, Pseudomonas aeruginosa, Staphylococcus aureus, Salmonella enterica, Staphylococcus epidermidis, Serratia marscensens or Streptococcus pneumoniae.

In at least some embodiments the species is a species of pathogenic microbe, in particular at least one of Acinetobacter baumannii, Enterococcus faecium, Candida auris, Escherichia coli or Staphylococcus aureus, for example one of Escherichia coli or Staphylococcus aureus.

The term “sample” refers to a collected variant of a species. A sample may be obtained, for example, from a sample source, such as a person, a hospital hallway, a ward, an intensive care unit ICU, or the like. Sample source refers to an entity from which (or from the surface of which) at least one sample may be collected. For example, hospital surfaces, treatment staff such as nurses and doctors, medical equipment such as stethoscopes and EKG machines, general equipment such as computers and mops may be sample sources. For example, patients may be sample sources. Further examples of a sample source include a healthcare worker, a medical device, an implement, or an environmental sample. An implement may be or comprise an item used in a medical setting, for example a cleaning tool such as a mop or broom, or for example a rubber glove.

As used in the context of the present disclosure, the term “mutation rate” refers to the rate at which a species, or strains and/or variants thereof, mutate over time. In other words, mutation rate may be defined as number of SNP differences over time, for example number of SNPs per month. In at least some embodiments, the mutation rate may be a function dependent species in question but also on at least one of: location, humidity, temperature, air composition or time. A typical mutation rate may be a literature value, or a statistical value for a species in question, for example.

In terms of epidemiology, transmission dynamics of viruses may be modelled using a compartmental model, such as a “Susceptible-(Exposed)-Infectious-Recovered”, S(E)IR, model. This is because viruses typically exhibit a finite period of infectivity, wherein the infectious disease caused by such a virus may be severe or fatal. However, over the course of time, such virus strains become less harmful, for example, due to mutations of said virus strain and a targeted host immune response. Thus, viral infections may exhibit an elevated period of infectivity, which is followed by a lowered period of infectivity and/or a period of inactivity. However, such S(E)IR models may not necessarily be feasible for other types of microbial strains, such as bacteria and fungi. As microbes, such as bacterial and fungal strains, may exhibit an elongated and/or indefinite window of infectivity, transmission dynamics may be difficult to model using such a S(E)IR, model, for example. Contrary to that of viral strains, bacterial and fungal strains may exhibit a continuous level of infectivity, such as a near constant level of infectivity. Moreover, for such microbes, antimicrobial resistance, AMR, may develop over time causing especially harmful implications in hospitals and healthcare. Such strains may lack a well-defined peak for infectivity making them harmful and long-lasting. Therefore, transmission timelines and transmission dynamics for bacterial and fungal species and strains may be more complex, and therefore more difficult to model and construct. Therefore, mutation of such species and transmission dynamics of such microbes should be known.

The term “tree” or “tree graph” as to be understood in the context of the present disclosure, is a model for interconnected relations of nodes (also known as vertices) connected via edges (also known as branches), such that each pair of nodes are connected via a respective edge. The term “node” refers to a point connected via edges, said point comprising, for example, sample genomic data. Nodes may comprise information such as sample genomic data and/or SNPs or information thereof. A tree may also be known as an acyclic graph. In other words, in a tree, edge relations are arranged recursively according to a tree data schema, being acyclic. A tree may be a “directed tree” (also known as an “oriented tree”), being thus, a directed acyclic graph, DAG. A directed tree is topologically ordered comprising edges having a direction and value, wherein an edge connects a pair of nodes. Examples of trees include phylogenetic trees, dated phylogenetic trees and updated phylogenetic trees. In general, an ordered tree comprises a parent node and child nodes thereof. Nodes may be sample nodes, representing a sample. Nodes may be internal nodes 265, for example non-sample nodes 264. While it is beneficial to represent each sample as a node in an ordered tree, it is also possible to exclude one or more samples from a tree. Such an exclusion may be done, for example, based on data integrity and/or data quality checks. Such exclusion may be part of rules 201.

The term “topology” refers to a property of a tree (graph) depicting relations of nodes and edges therein, and in particular number, arrangement and positioning of said nodes and edges. Therefore, a topology of a tree is at least based on connectivity of nodes, hierarchy of nodes, uniqueness of edges, and number of nodes. A tree may be defined to be topologically less complex if it comprises at least one of: fewer nodes or fewer edges, than another (more complex) tree.

In accordance with the present disclosure, there is provided a method for obtaining a transmission timeline. In said method, SNP information from a sample, mutation rate of the species and timestamp for the sample collection are used to generate a dated phylogenetic tree and a transmission timeline. The SNP information may be obtained by comparing a reference genome to sample genomic data obtainable from a collected sample. For the dated phylogenetic tree, a tree updating process may be performed so as to provide a transmission timeline comprising information on the transmission dynamics of the species. Therefore, the term “transmission timeline” comprises evolutionary information and connections between genomic and/or genetic variants of a species wherein said variants may differ with respect to one or more SNPs from one another. Furthermore, such a timeline may be used to model, track and analyse outbreaks and transmission dynamics of species.

SNP information may be obtained from sample genomic data. Sample genomic data is a digital representation of a genomic DNA sequence of a sample of the species in question. In accordance with the present disclosure, in at least some embodiments, said sample genomic data is compared to a reference genome of the species in question. Comparison may be done, for example, using SNP calling. The term “reference genome” as used in the context of the present disclosure is to be understood as a genome or part of a genome, of a strain of a species to which sample genomic data may be compared. The reference genome may be, for example, a genome or part thereof of a “common ancestor” of strains/variants of samples in question. A common ancestor refers to a sample, or sample genomic data thereof, to which newer samples may be connected directly or indirectly to other samples and sample genomic data thereof via edge relations. As can be appreciated by a person skilled in the relevant art, a reference genome may be obtained in a similar way as the sample genomic data. In at least some embodiments, the reference genome is used as an “anchor” to align the samples' genomes to each other.

In order to obtain information of SNPs of a collected sample, SNPs of sample genome of a species are compared to a reference genome of the species. From the comparison, SNP information is obtained representing SNP(s) of the corresponding sample. Such information may comprise at least one of: position or value of the SNPs. In at least some embodiments, SNP information may be a vector, or a matrix. In at least some embodiments, SNP information may be a list of 2-tuples, depicting substituted nucleotide and position of SNPs within sample genomic data with respect to the reference genome. In at least some embodiments, the SNP information comprises: the SNP position in the reference genome, original nucleotide of the reference genome, and the mutated nucleotide of the sample.

“Mutated nucleotide” refers to the nucleotide substituted in sample genomic data, and therefore differs from the nucleotide in the same position of the reference genome. “Original nucleotide” or “reference nucleotide” refers to the nucleotide of the reference genome to which sample genomic data and SNPs therein are compared. Therefore, mutated nucleotide is different from the corresponding reference nucleotide. In at least some embodiments, the SNP information may be presented by 3-tuples depicting substituted nucleotide, original nucleotide and position of SNPs within sample genomic data with respect to the reference genome.

The genetical difference may be used to estimate the evolutionary relationship of sample genomic data and a reference genome, for example. The term “genetically different” is to be understood as differences in two or more genetic material, such as a genome or part thereof. Such a two or more genetic materials may be different, for example, in terms of single nucleotide polymorphisms, SNPs. The comparison of sample genomic data with a reference genome may be done in view of SNPs within the sample genomic data with respect to the reference genome. Therefore, the sample genomic data may be compared to the reference genome with respect to SNP substitutions, for example, in terms of position of the SNPs and/or variations in nucleotides along the sample genomic data. Alternatively or in addition, SNPs of sample genomic data from two or more samples may be compared.

The reference genome should be selected such that it provides a fair representation of the species and its historical lineage. In other words, a too young reference genome may hinder analysis, and fail to provide a fair representation, because such a reference genome may fail to represent a common ancestor of the sample or plurality of samples, for example. As such, a common ancestor of the sample or a plurality of samples may be a suitable candidate for the reference genome.

From at least the SNP information, SNP evolutionary distance may be determined. “SNP evolutionary distance” is to be understood as a difference between two strands of genomic data, for example, a difference between sample genomic data and a reference genome. Therefore, SNP evolutionary distance may be between sample genomic data and the reference genome. Alternatively or in addition, SNP evolutionary distance may be between sample genomic data of a plurality of samples. As such, the SNP evolutionary distance may be obtained by comparing the SNPs between the sample genomic data from two or more samples or by comparing the SNPs of sample genomic data to a reference genome, for example. SNP evolutionary distance is depicted typically as an integer. The SNP evolutionary distance depicts the relative distance in terms of SNPs between two nodes, such as a sample node and an internal node connected thereto, or such as a sample node and another sample node. The SNP evolutionary distance describes the difference between two strands with respect to the position and value of the nucleotides of SNPs therein as well as the number of different SNPs.

The determining of SNP evolutionary distance may be conducted by computing the difference of SNP information of two or more sample genomic data, or computing the difference of SNP information of sample genomic data to that of a reference genome. In at least some embodiments, computing the difference of SNP information of a first sample genomic data and SNP information of a second genomic data provides the overall difference, similarity or dissimilarity of SNPs, for example as an integer value depicting the number of differences in SNPs between said first sample genomic data and the second sample genomic data.

For example, given a first sample genomic data having a sequence “ACGTACG” and a second sample genomic data having a sequence “GCGTACA”. Because the first and last nucleotides of second sample genomic data differs from the first (i.e., two differences in total), the SNP evolutionary distance of the first and second sample genomic data is “2”. Similarly, if the second sample genomic data is construed to be a reference genome, the SNP evolutionary distance of the first sample genomic data to the reference genome is “2”. In another example, sample genomic data is “GGGGGGGG” and a reference genome is “GGGGGAAA”, the difference, and SNP evolutionary distance is “3”.

In at least some embodiments, genomic sequencing data may be compared to one or more reference sequences, such as a reference genome, in order to identify and locate the presence or absence of SNPs in said genomic sequencing data. Sample genomic data may comprise genomic sequencing data. The information on absence, i.e., the negation of presence, of SNPs provides a closer similarity of the genomic sequencing data to the one or more reference sequences than another genomic sequencing data having more SNPs present with respect to the one or more reference sequences.

A mutation rate may be obtained from experimental data, statistical data or literature values, for example. In at least some embodiments, the mutation rate may be estimated from the SNP information of a plurality of samples and corresponding timestamps. The mutation rate may be, for example, 1 SNP/month. In at least some embodiments, information on humidity and temperature conditions of a sample source may be utilized at least for obtaining a mutation rate and/or adjusting the mutation rate.

SNP information and corresponding sample collection timestamp as well as mutation rate of the species may be used at least in part to obtain a dated phylogenetic tree.

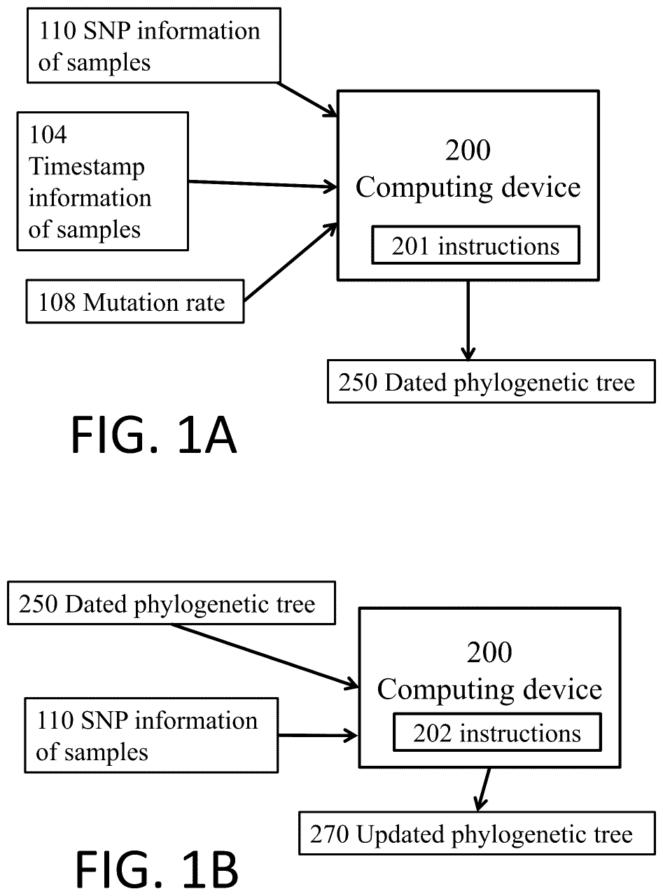

FIG. 1A illustrates an example embodiment comprising a computing device 200 configured to obtain a dated phylogenetic tree 250 from SNP information 110 of a plurality of samples, corresponding timestamps 104 and a mutation rate 108 corresponding to the species in question. The dated phylogenetic tree 250 is generated by the computing device 200 using instructions 201. In other words, said computing device 200 is configured to generate a dated phylogenetic tree 250 based on obtained SNP information 110, corresponding timestamp 104 and mutation rate 108. The computing device may be a computing device comprising a processor, for example a server. The server may be configured to participate in providing a cloud service, for example.

The computing device, such as the computing device 200 of FIG. 1A, is configured to generate a dated phylogenetic tree, wherein samples are transformed into sample nodes at least in part connected via edges. The dated phylogenetic tree is generated using at least the SNP information and the mutation rate. SNP information of a sample describes the SNPs of the sample of the species in relation to the reference genome of the species.

In addition to the SNP information, the mutation rate and timestamps, the dated phylogenetic tree may be generated using additional information. Additional information may comprise one or more of: locations of sample sources and public data, for example. Public data may be, for example, information on reference genome and/or publicly distributed sample genomic data. Location of sample sources may be referred to as “swim lanes”, especially when samples are arranged by location.

In at least some embodiments, the dated phylogenetic tree may be updated so as to obtain an updated phylogenetic tree. FIG. 1B illustrates an example of updating a dated phylogenetic tree 250. In FIG. 1B, a computing device 200 is configured to receive the dated phylogenetic tree 250 and optionally SNP information 110 of samples. Moreover, said computing device 200 is configured to use updating instructions 202 in order to generate an updated phylogenetic tree 270. In at least some embodiments, the updated phylogenetic tree may be generated directly after the corresponding dated phylogenetic tree.

A computing device, such as a computing device 200 FIGS. 1A & 1B, may be configured to construct and/or display a visual representation of the dated phylogenetic tree and/or an updated phylogenetic tree. For example, such a visual representation may be displayed in a web browser.

In at least some embodiments, prior to construction of a dated phylogenetic tree, a phylogenetic tree may be constructed. The term “phylogenetic tree” refers to connections between variants of a species in terms of genomic differences, for example between a common ancestor and variants thereof. The connections may refer to differences between variants in terms of SNPs. Phylogenetic tree, however, fails to depict the direction of evolution as such. In other words, a phylogenetic tree, and nodes therein, is time-independent. In a phylogenetic tree, sample nodes are typically leaves.

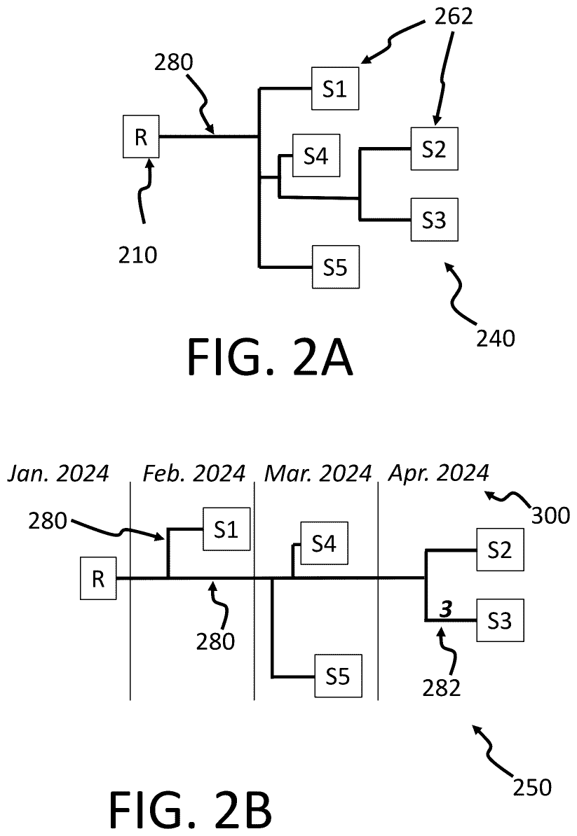

FIG. 2A illustrates an example phylogenetic tree 240, having a common ancestor 210 (designated with “R”, because it is the root of the tree), and sample nodes 262, (designated with “S1”, “S2”, “S3”, “S4” and “S5”) connected via edges 280 to at least one other node, for example the common ancestor 210. For the sake of clarity, only sample nodes S1 and S2 have reference number 262 within FIG. 2A, but all samples S1-S5 are sample nodes within the figure. As can be appreciated from FIG. 2A, such a phylogenetic tree 240 lacks temporal information, and such a construction is dependent on differences in SNPs of samples. In other words, such a phylogenetic tree fails to provide temporal evolution of samples.

On the other hand, a “dated phylogenetic tree”, also known as a “timed tree”, refers to a tree comprising historical (temporal) and evolutionary connections between variants of a species and differences in SNPs between said variants. The dated phylogenetic tree is thus, a directed tree, wherein a temporal and evolutionary connections of nodes are provided as edge relations, and relative positions of nodes.

A dated phylogenetic tree may be generated based on SNP information, SNP evolutionary distances, timestamp and generation rules. The term “generation rule” or plurality thereof refers to instructions, which may be used in part to generate a dated phylogenetic tree. Generation rules may comprise a plurality of rules, for example any one of the following exemplary rules:

A generation rule may comprise instructions, that when executed, generate sample nodes comprising SNP information, each sample node having the timestamp of the respective sample. Further, a generation rule may provide instructions, that when executed, compare SNP information and/or SNP evolutionary distance of sample genomic data from a plurality of samples with one another. A generation rule may comprise instructions, that when executed, compare sample genomic data from a plurality of samples to a reference genome. Based on, for example, such a comparison, a dated phylogenetic tree is constructed by connecting sample nodes to one another having a minimum SNP evolutionary distance via an edge. Moreover, generation rules may infer non-sample nodes as internal nodes depicting an ancestral node for one or more sample nodes. By performing the generation rule or a plurality of generation rules, a dated phylogenetic tree is obtained. Thus, a generation rule, such as instructions 201 of FIG. 1A, includes an instruction at least in part using which the transmission timeline may be obtained.

An edge of a dated phylogenetic tree may have a weight, for example a SNP evolutionary distance. The term “child node” in the context of a dated phylogenetic tree or an updated phylogenetic tree refers to a node connected to a parent node, said child node being historically newer than said parent node. Conversely, “parent node” refers to a node connected to a child node, said parent node being historically older than said child node.

The term “sample node” refers to a node information of a tree, such as a phylogenetic tree, dated phylogenetic tree or an updated phylogenetic tree, which is obtained from a sample. In other words, sample node is generated from SNP information obtained from sample genomic data of a sample. On the other hand, the term “non-sample node” refers to a node, information of which is inferred, computed or implicitly obtained and/or deduced at least in part from a sample, or plurality thereof. Therefore, the term “non-sample” refers to information implicitly obtained from statistical data and collected samples, for example. For example, a non-sample may be an inferred ancestor to collected samples, based on mutation rate of the species, timestamp(s) of the sample(s) and SNP information of the sample(s). A non-sample node may be a “transmission origin” or a “lineage point”, for example. Transmission origin refers to a non-sample node from which a plurality of lineages may diverge. A lineage point refers to a non-sample node to which one or more sample nodes or non-sample nodes are connected as child nodes. Lineage point represents a closest common ancestor for two or more sample nodes. In other words, the two or more sample nodes, are connected to said lineage point via separate edge relations.

FIG. 2B illustrates an example of a dated phylogenetic tree 250. The dated phylogenetic tree is constructed from nodes and edges 280 connecting two of such nodes. In FIG. 2B, sample nodes are depicted with reference signs S1, S2, S3, S4 and S5. In FIG. 2B, a timeline 300 for sample nodes, and therefore for samples, is depicted. As can be appreciated in FIG. 2B, the timeline may highlight the temporal connections of samples to one another. Therefore, the inferred differences in SNPs and timestamps of sample provide a dated phylogenetic tree.

A dated phylogenetic tree describes transmission dynamics and evolution of a species with temporal information. Such information is not present in a phylogenetic tree, such as the phylogenetic tree illustrated in FIG. 2A. For example, in FIG. 2B, sample S1 and information thereof may be used in analysis of transmission dynamics, as the temporal position is known. In other words, a timepoint at which such difference in SNPs occurred is provided. Moreover, an inferred parent and corresponding timepoint thereof may be obtained. Conversely, FIG. 2A sample node S1 fails to provide such information.

The SNP evolutionary distance may be defined with respect to the common ancestor (such as a reference genome), or between a parent node and a child node. Node-to-node SNP evolutionary distance is to be understood to refer to the difference in terms of SNPs between two nodes connected via an edge. A weight 282 is shown within FIG. 2B having an exemplary value of 3, where 3 is the SNP evolutionary distance between S3 and the previous junction of edges.

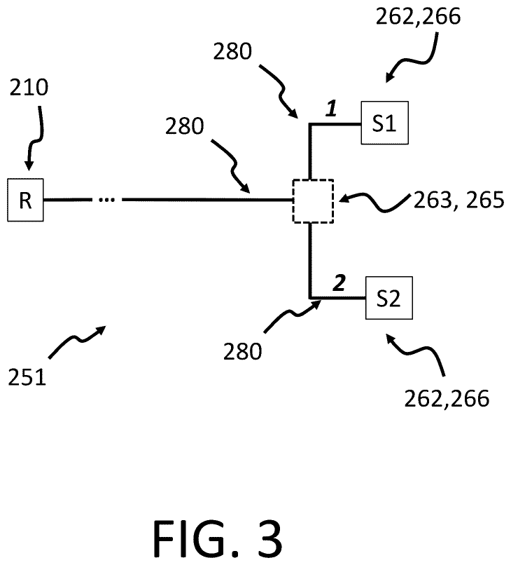

FIG. 3 illustrates a simplified dated phylogenetic tree 251 comprising sample nodes 262 (designated with “S1” & “S2”), as well as an internal node 263 as a parent node 265 thereof. In other words, sample nodes 262 are child nodes 266 of said internal node 263. Moreover, the internal node 263 is directly or indirectly connected to a common ancestor 210 (designated with “R”). In FIG. 3, the internal node 263 is depicted with a rectangle having a dashed line perimeter. Such an internal node may be a non-sample node. In other words, sample nodes S1 & S2 are connected via respective edges 280 to a non-sample node which is the evolutionary parent or ancestor of said sample nodes.

As can be appreciated form FIG. 3, the edges depict numerical SNP evolutionary distance between respective connected nodes. The sample node S1 is derived from a sample having 1 SNP evolutionary distance from the internal node, whereas sample node S2 is derived from a sample having 2 SNP evolutionary distance to said internal node. Moreover, the SNPs with respect to the internal node, are different between sample node S1 and sample node S2: As sample node S2 is not a child node of sample node S1 in the example of FIG. 3, the SNPs of said sample nodes are different from one another. Furthermore, samples corresponding to sample node S1 and sample node S2 have been collected at (near) same time, due to the distance of the sample nodes S1&S2 to the parent node 265. In other words, the depicted horizontal direction refers to time, wherein earlier time points are depicted on the left side, and later time points are depicted on the right.

In a dated phylogenetic tree, sample nodes may be connected via internal nodes comprising non-sample data inferring a common ancestor for one or more such sample nodes acting as child nodes thereof. A node of a dated phylogenetic tree may be an internal node or a sample node. An internal node of a dated phylogenetic tree may be a non-sample node, and such an internal node may depict a lineage point or a transmission origin. The internal node is an unsampled genetic ancestor of child nodes connected via edges thereto.

The edge connecting two nodes provides at least the SNPs evolutionary distance between said two nodes. In at least some embodiments, an edge may depict at least two values: time difference and SNP evolutionary distance between two nodes. In other words, the value of the edge is the difference in SNPs between two nodes connected therewith. Thus, the dated phylogenetic tree may comprise a plurality of lineages of variants for the species in question. In at least some further embodiments, information on sample collection location, such as a sample source, may be used in the obtaining of the dated phylogenetic tree.

In at least some embodiments, edge relations may comprise a distance depicting temporal displacement of sample and/or inferred time of mutation. Moreover, the edge relations may comprise a value of the SNP evolution distance between the nodes connected via said edge. Thus, an edge may be represented as a 2-tuple. Such a 2-tuple may be, for example, at least one of (time, SNP evolutionary distance) or (distance with respect to time, SNP evolutionary distance).

The dated phylogenetic tree provides evolutionary relationships between a plurality of samples of the species based on genomic information and timestamps of each sample in the plurality of samples. In at least some embodiments, a mutation rate may be estimated at least in part from the timestamps of collected samples and corresponding SNP information obtained thereof.

For a generated dated phylogenetic tree, an tree updating process may be conducted thereby generating an updated phylogenetic tree. The topology of an updated phylogenetic tree may be less complex than that of the corresponding dated phylogenetic tree. In the tree updating process, common ancestors for the sample nodes are re-evaluated. During the tree updating process, the need for an internal node connected to one or more sample nodes is evaluated based on at least the SNP evolutionary distance and an updating rule or a plurality thereof. Therefore, using updating rules may provide a less complex topology for a transmission timeline and a model for transmission dynamics.

An example of an updating rule includes the following. Given a scenario wherein one or more sample nodes is a child node of an internal node (i.e., said internal node is a parent node), in a case where the SNP evolutionary distance of one of the child nodes to the parent node is null, the parent node is deleted, and the sample node in question is promoted as a new parent node. As such, other possible child nodes of the internal node in question are attached to the new parent node via corresponding edges. Thus, an updated phylogenetic tree is obtained wherein excessive internal nodes are removed and/or transformed into sample nodes. Contrary to a dated phylogenetic tree, an internal node may thus, be a sample node.

At least in connection with the updating rule provided above, another example of an updating rule may be as follows. Given a scenario wherein one or more sample nodes is a child node of a non-sample node (internal node), in a case where a timestamp of a sample node does not follow the mutation rate for the species, promotion of said sample node is left undone. The following example provides one applicable scenario for the updating rule above. In the scenario, an internal node (parent node) is dated 01.01.2024 (dd.mm.yyyy), a first sample node (a first child node) is dated 01.02.2024 and a second sample node (a second child node) is dated 02.02.2024, and the first child node has 0 SNP evolutionary distance to the parent node and second child node 5 SNP evolutionary distance to the parent node. In such a scenario, the internal node may be kept as the parent node, because promotion of the first child node to a new parent node would infer a mutation rate of 5 SNPs per day, an exceedingly high mutation rate of the species in question.

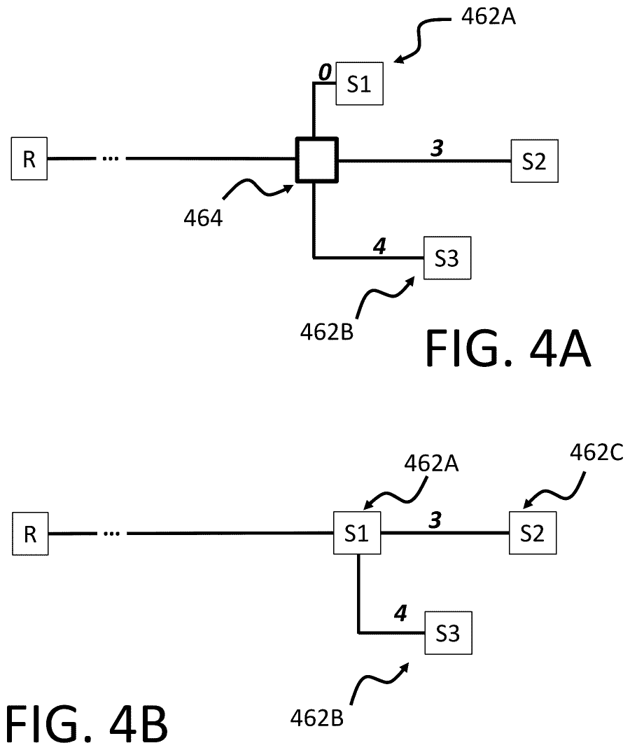

An example of a promotion in a dated phylogenetic tree is illustrated in FIGS. 4A & 4B, wherein a promotion of a child node to a parent node occurs. In other words, FIG. 4A illustrates an example phylogenetic tree prior to promotion and FIG. 4B a corresponding phylogenetic tree succeeding said promotion of a child node.

FIG. 4A depicts a dated phylogenetic tree wherein edges represent SNP evolutionary distance and time difference between said two respective nodes. In FIG. 4A, three sample nodes 462A,462B & 462C are presented as “S1”, “S2” & “S3”, respectively. Moreover, said sample nodes are connected to a non-sample node 464 acting as an internal node. In FIG. 4A, sample node S1 has a null SNP evolutionary distance (“0”) to an internal node acting as a parent node. Moreover, a sample node S2 and a sample node S3 have SNP evolutionary distance of 3 and 4, respectively, to the parent node.

FIG. 4B depicts a dated phylogenetic tree derivable from dated phylogenetic tree of FIG. 4A. In other words, sample nodes S1, S2 & S3 correspond to sample nodes depicted in FIG. 4B. In FIG. 4B, the sample node S1 has been promoted to a parent node (i.e., in this case replacing internal node) based on its SNP evolutionary distance. Moreover, sample nodes S2 and S3 have been attached to sample node S1 via corresponding edges. The position (time of timestamp) of S1 does not change as a result of the promotion. In other words, in FIG. 4B the sample node S1 is the (promoted) parent node of the sample nodes S2 & S3.

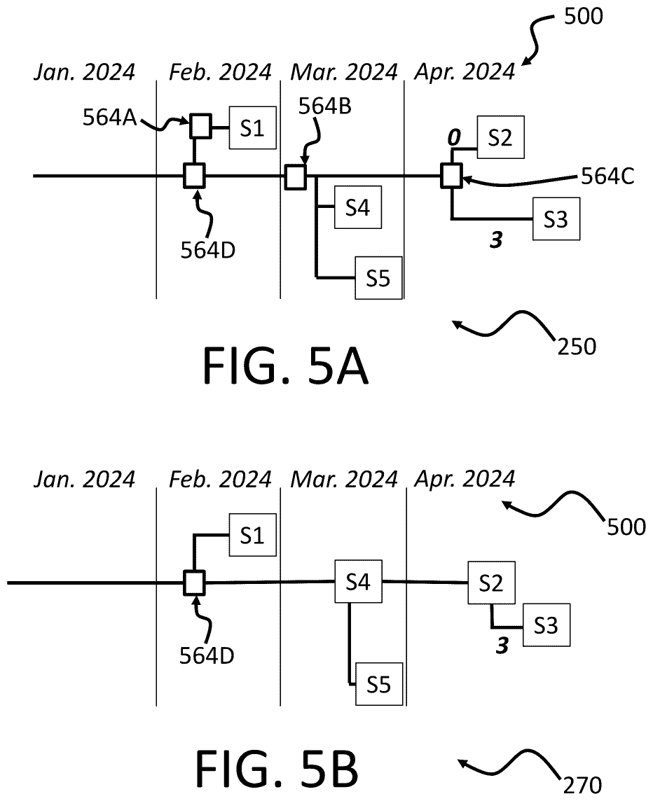

FIGS. 5A & 5B illustrate an example dated phylogenetic tree 250 and updated phylogenetic tree 270, respectively. In FIGS. 5A & 5B, a timeline 500 is presented wherein time progresses from January 2024 to April 2024 (i.e., from left to right in the illustration of FIGS. 5A & 5B). Any reference genome, or common ancestor (not shown in figure) would predate January 2024. Moreover, sample node S1 has been collected in February 2024, while sample S4 and sample S5 have been collected in March 2024, and while sample S2 and S3 have been collected in April 2024. Said samples have been collected and their genomic data extracted and processed so as to provide sample genomic data and SNP information. Non-sample nodes of FIGS. 5A and 5B are depicted as blank rectangles.

Referring to FIG. 5A, wherein internal nodes 564A, 564B, 564C & 564D and sample nodes S1, S2, S3, S4 & S5 are presented. Said nodes are connected to one or more other nodes via edges. In FIG. 5A, sample node S2 has null SNP evolutionary distance (“0”) to parent node 564C. Sample node S3 has SNP evolutionary distance of 3 to said internal node 564C. Samples S2 and S3 are timestamped so that S2 is 1 day earlier than S3, which is reflected by the x-axis placement of the samples. Sample node S4 has a SNP evolutionary distance of 0 to internal node 564B (distance not marked in figure for sake of clarity). Sample node S1 is connected to an internal node. Non-sample nodes 564A-564D of FIG. 5A may refer to a common ancestor, said common ancestor having inferred SNPs based on sample nodes, for example parent and child sample nodes of the non-sample node.

FIG. 5B depicts an updated phylogenetic tree 270 wherein excess internal nodes 564A, 564B & 564C have been removed, and topology of the tree thereby corrected. The updating process may comprise performing ancestral reconstruction for each internal node, and then promoting the sample nodes that exactly match their reconstructed parent (i.e. which have a 0-SNP difference to the their parent's reconstruction). Further, excess internal nodes may be removed. Ancestral reconstruction typically refers to inferring the most likely SNPs of each internal node, without doing any changes to tree topology. A dated phylogenetic tree which has undergone the updating process may be known as an updated phylogenetic tree.

In FIG. 5B, it can be seen that the sample S4, having an SNP distance 0, has been promoted. The same applies to S2. FIG. 5B therefore illustrates 2 update operations (that of S2 and S4). A possible exception to promotion is when a promotion would imply a evolution (measured in SNP distance) which is too rapid in comparison with the timestamps. (for example a 3-SNP evolution within 2 hours). The updating rules take such exceptions into account.

While FIGS. 2A, 2B, 3, 4A, 4B, 5A & 5B depict examples of trees and properties thereof, such figures are mere example illustrations of the trees provided in accordance with the present disclosure. As a person skilled in the art readily appreciates the actual tree, such as a dated phylogenetic tree and/or an updated phylogenetic tree are stored in computer memory and manipulated therein. As such, the entities depicted in figures, although factual, should be construed as visual aids in assisting to understand at least some of the embodiments of the present disclosure.

In order to obtain sample genomic data and SNP information in accordance with at least some embodiments of the present disclosure, sample or plurality thereof may need to be collected. In the following, examples of sample collection, pre-processing and comparison are provided so as to obtain such sample genomic data and SNP information.

In at least some embodiments, a sample is collected together with a corresponding timestamp indicating a collection time of said sample. Such a phase may be known as a sample collection phase. As used in the context of the present disclosure, the term “timestamp” refers to time at which a sample has been collected from the sample source, for example, in terms of year, month, day, hour and/or minute.

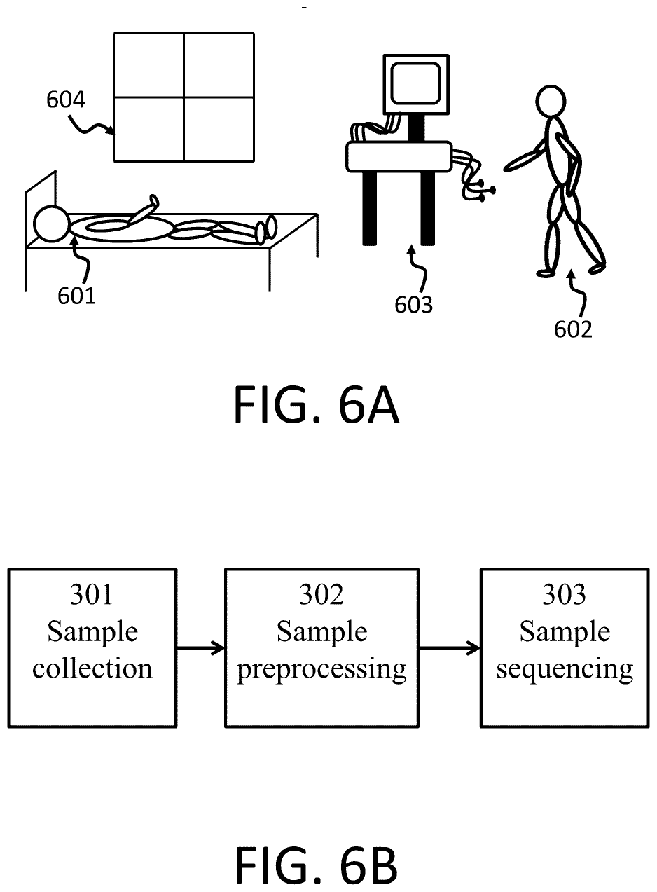

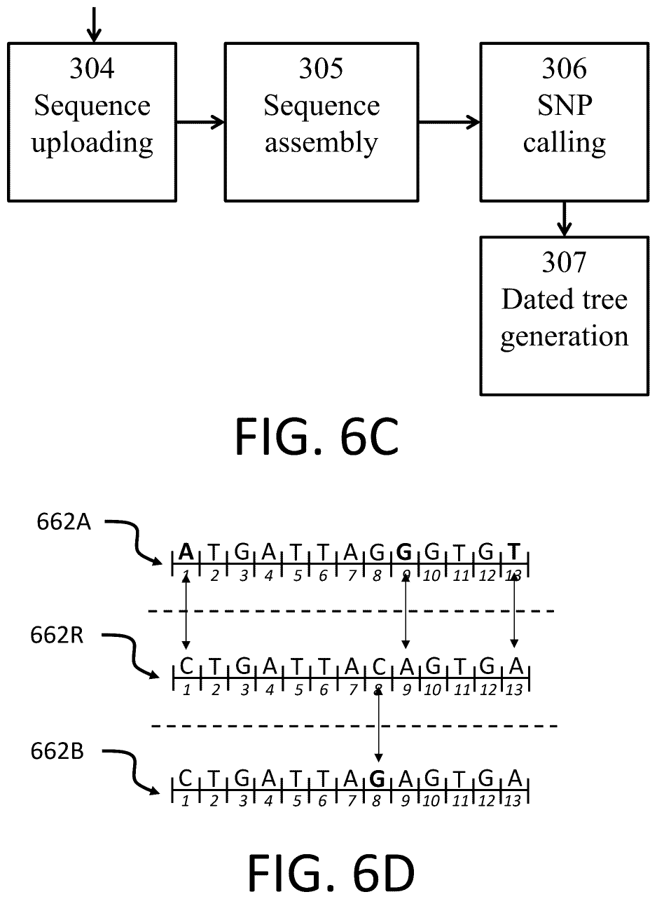

FIG. 6A illustrates an example of sample sources for sample collection. In FIG. 6A, four different sample sources are presented: a patient 601, hospital staff 602, hospital equipment 603 and a surface 604.

FIGS. 6B & 6C illustrate an example method in accordance with the present disclosure. In step 301, a sample is collected, for example, from locations disclosed elsewhere in the present disclosure, for example one or more of 601, 602, 603 or 604 of FIG. 6A. Typically, a plurality of samples of the species are collected, said collection of plurality of samples differing in terms of time and/or location. The plurality of samples may be collected from different locations, such as hospital hallways, hospital wards, and intensive care units. In at least some embodiments, a sample may be obtained from a patient or a sample may be an environmental sample. In at least some embodiments, such a sample may be known or suspected to comprise a pathogenic microbe.

Then, in step 302, a sample is pre-processed. Such a pre-processing may comprise an extraction and/or isolation phase, for example. In an extraction phase, genetic material is extracted from the sample. In other words, strands of DNA are extracted and isolated from rest of the structures for further processing. The sample comprises genetic material. The term “genetic material” is to be understood as DNA extracted from a sample. In at least some embodiments, a pure culture from a sample is prepared. From said prepared pure culture, genomic DNA may be isolated thereby obtaining genetic material or strands thereof.

In step 303, strands of genetic material are sequenced. Such a step may be known as a sequencing phase. In such a sequencing phase, sequence reads of the genetic material are obtained. The obtained sequence reads may be known as “reads”. The term “read” or “a sequence read” refers to a sequence of nucleotides or base pairs corresponding to all or part of a single genetic material fragment. Therefore, a digital representation of the sample genomic data is obtained in the form of a read.

In at least some embodiments, the genetic material of the sample may be sequenced into smaller portions or fragments, also known as “short reads”. In other words, short reads are obtained via fragmentation. However, in at least some embodiments, “long reads” are also possible depending, for example, on the available hardware and instrumentation. Long reads refer to longer portions or longer genetic fragments of the genetic material for which fragmentation has not been necessarily conducted. After obtaining the sequence reads, said reads are uploaded to the digital platform. In other words, the sequence reads are digitized and uploaded to an electronic device for further processing.

Referring then to step 304 of FIG. 6C, the sequence reads are uploaded to a computing device, such as a server.

In step 305 of FIG. 6C, at the computing device, sample genomic data of the sample is obtained. In at least some embodiments, for example in which short reads are used, an assembly phase is conducted, wherein the sequence reads are digitally assembled and/or combined into longer sequence portions forming thereby a sample genomic data of the sample. Long reads may also be assembled. In the assembly phase, the whole genome, or parts thereof, is obtained as a digital representation of a strand of genetic material, known as sample genomic data. Thus, such sample genomic data refers to a genome assembly. A genome assembly, also known as sample genomic data, may be obtained from fragments of a sample genomic data by matching and combining corresponding ends of the fragments of DNA.

In step 306 of FIG. 6C, SNP calling is conducted for said sample genomic data. SNP calling is a technique for comparing sample genomic data to a reference genome in order to obtain SNP information. In SNP calling a sample genomic data is aligned to a reference genome in order to identify nucleotides in which said sample genomic data differs from the reference genome. In other words, SNP value and location in the sample genomic data may be obtained using SNP calling.

In step 307 of FIG. 6C, a dated phylogenetic tree is generated. The dated phylogenetic tree may be generated by a computing device, such as the computing device 200 of FIG. 1A. Moreover, in step 307, at least the SNP information is used to generate the dated phylogenetic tree. SNP information may be obtained from sample genomic data via comparison to a reference genome, using SNP calling, for example.

FIG. 6D illustrates an example of comparison of sample genomic data 662A of a first sample, a reference genome 662R and sample genomic data 662B of a second sample. In FIG. 6D, sample genomic data 662A & 662B and reference genome 662R depict a matching portion of the reads, each being 13 nucleotides in length. As a person skilled in the art is aware, other lengths and permutations are applicable, and provided sequences are mere examples of SNP calling. In at least some embodiments, such a comparison of sample genomic data of a plurality of samples occurs concurrently, while in some other embodiments, comparison of sample genomic data of a plurality of samples are sequential.

In the example illustrated in FIG. 6D, the sample genomic data 662A and the sample genomic data 662B are compared to the reference genome 662R. With such comparison, SNP information for said sample genomic data 662A and sample genomic data 662B are obtained. For example, as can be appreciated from FIG. 6D, sample genomic data 662A differs from the reference genome by 3 SNPs. Said 3 SNP differences are at positions 1 (“A”), 9 (“G”) and 13 (“T”) as read starting from the left side of the sample genomic data 601A. Moreover, sample genomic data 662B differs from the reference genome by 1 SNP. Said 1 SNP difference is at position 8 (“G”) as read starting from the left side of the sample genomic data 662B.

As a simplified example based on SNP calling of FIG. 6D, the obtained SNP information may therefore comprise 2-tuples (“A”,1), (“G”,9) and (“T”,13) for sample genomic data 662A, and a 2-tuple (“G”, 8) for sample genomic data 662B. Thus, the SNP evolutionary distance of the first sample to the reference genome is 3, and the SNP evolutionary distance of the second sample to the reference genome is 1. Moreover, the SNP evolutionary distance of the first sample to the second sample is 4, because the 2-tuple of SNP information from sample genomic data 662B differs with respect to every 2-tuple of SNP information from sample genomic data 662A.

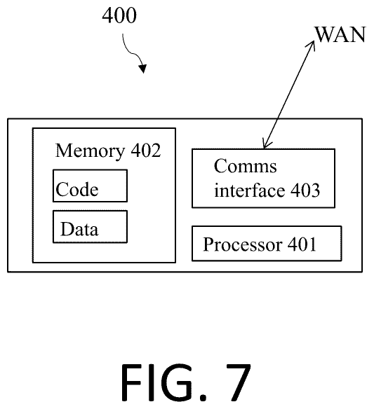

FIG. 7 illustrates an example apparatus 400 capable of supporting at least some embodiments of the present disclosure. The apparatus 400 comprises at least one processor 401, and at least one memory 402 including computer program code, and optionally data. The apparatus 700 may further comprise a communication unit or interface (“Comms interface” 403). Such a unit may comprise, for example, a wireless and/or wired transceiver. The communication unit or interface 403 may be connected to a wide area network, WAN, for example the internet. WAN may be used to obtain digitized reads of DNA, for example. The apparatus 700 may also include other elements not shown in FIG. 7.

In at least some embodiments, the apparatus 400 may be, or correspond to, computing device 200 of FIG. 1A and/or FIG. 1B. Although the apparatus 700 is depicted as including one processor, the apparatus 700 may include more processors. In an embodiment, the memory is capable of storing instructions, such as at least one of: operating system, various applications, models, neural networks and/or, preprocessing sequences. Such instructions may be instructions 201 and/or instructions 202 of computing device 200 of FIGS. 1A &1B. Furthermore, the memory may include a storage that may be used to store, e.g., at least some of the information and data, such as sample genomic data, SNP information and/or SNP evolutionary distance, used in the disclosed embodiments.

Furthermore, the processor is capable of executing the stored instructions. In an embodiment, the processor may be embodied as a multi-core processor, a single core processor, or a combination of one or more multi-core processors and one or more single core processors. For example, the processor may be embodied as one or more of various processing devices, such as a processing core, a coprocessor, a microprocessor, a controller, a digital signal processor (DSP), a processing circuitry with or without an accompanying DSP, or various other processing devices including integrated circuits such as, for example, an application specific integrated circuit (ASIC), a field programmable gate array (FPGA), a microcontroller unit (MCU), a hardware accelerator, a special-purpose computer chip, or the like. In an embodiment, the processor may be configured to execute hard-coded functionality. In an embodiment, the processor is embodied as an executor of software instructions, wherein the instructions may specifically configure the processor to perform at least one of the models, sequences, algorithms and/or operations described herein when the instructions are executed.

The memory may be embodied as one or more volatile memory devices, one or more non-volatile memory devices, and/or a combination of one or more volatile memory devices and non-volatile memory devices. For example, the memory may be embodied as semiconductor memories (such as mask ROM, PROM (programmable ROM), EPROM (erasable PROM), flash ROM, RAM (random access memory), etc.).

The at least one memory and the computer program code may be configured to, with the at least one processor, cause the apparatus 700 to at least perform as follows: obtain a reference genome for a species; obtain a timestamp for each sample of a plurality of samples, wherein at least one sample is from said species; obtain sample genomic data corresponding to each sample; obtain based on a difference between the reference genome and each sample genomic data, SNP information from each sample in the plurality of samples; obtain a mutation rate for said species; determining, based on the SNP information, SNP evolutionary distance from the reference genome for each sample; determine, based on the SNP information, SNP evolutionary distance between each sample; generate, based on the SNP information, the SNP evolutionary distances from the reference genome, the SNP distance between the samples, the mutation rate, generation rules and the corresponding timestamps, a dated phylogenetic tree, said tree comprising sample nodes and non-sample nodes, and wherein each sample corresponds to a node.

Apparatus 700 may be configured to generate a dated phylogenetic tree. In at least some embodiments, apparatus 700 may be further configured to update said dated phylogenetic tree thereby generating an updated phylogenetic tree. Apparatus 700 may be configured to at least participate in generating a dated phylogenetic tree, for example by performing any of the methods described elsewhere in the disclosure. Apparatus 700 may be configured to at least participate in providing a visual representation of a generated phylogenetic tree, for example a dated phylogenetic tree.

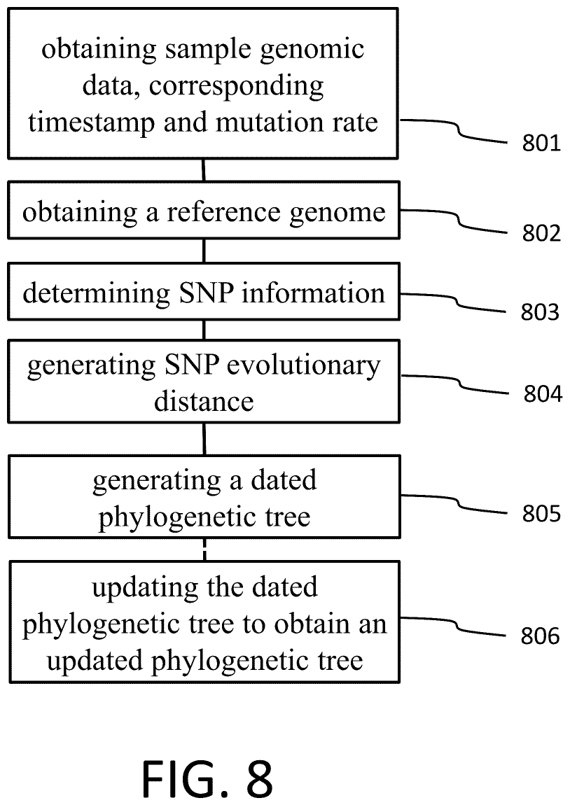

FIG. 8 illustrates a method in accordance with the present disclosure. The method may be at least in part executed by an apparatus, such as apparatus 700 of FIG. 7. Said method of FIG. 8 comprises steps 801, 802, 803, 804, 805 and 806.

In step 801, sample genomic data and corresponding timestamp as well as a mutation rate of the species is obtained. Sample genomic data and corresponding timestamp may be obtained from a plurality of samples. In step 802, a reference genome is obtained. In step 803, SNP information is determined at least based on the reference genome and sample genomic data. Then, in step 804, SNP evolutionary distance of sample genomic data is obtained with respect to the reference genome and possible other sample genomic data. In step 805, a dated phylogenetic tree is generated, based at least on SNP information, mutation rate and the timestamp. In step 806, the dated phylogenetic tree is updated using updating rules, such as rules 202 of FIG. 2B, in order to obtain an updated phylogenetic tree.

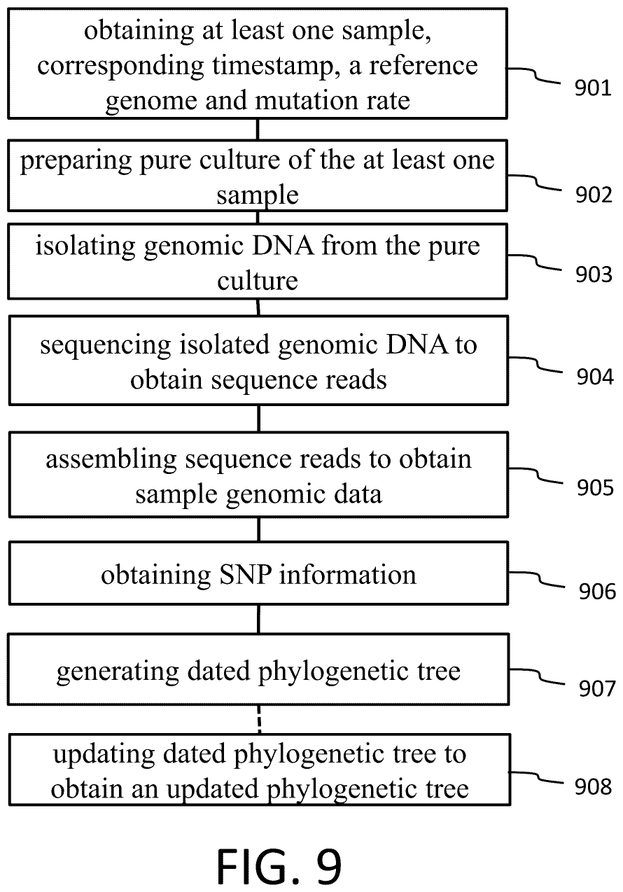

FIG. 9 illustrates a method in accordance with the present disclosure, said method comprising steps 901, 902, 903, 904, 905, 906, 907 and 908.

In step 901, at least one sample is obtained together with a corresponding timestamp. A reference genome and mutation rate for the species in question is obtained. The sample may be a pathogenic microbe, for example. In step 902, a pure culture of the at least one sample is prepared. In step 903, preparation of the pure culture, genomic DNA from the pure culture is isolated. Steps 901 to 903, therefore provide a pre-processed isolated genomic DNA.

In step 904, the isolated genomic DNA is sequenced thereby obtaining sequence reads. Such sequence reads are a digital representation of the fragments of the genome of the sample. In step 905, the sequence reads are assembled. Digital representation of the sequence reads may be thus, uploaded to a computing device, for said assembly. The assembled genome is also known as sample genomic data. The sample genomic data may comprise genomic sequencing data. In step, 906, SNP information is obtained using the sample genomic data. Therein, said sample genomic date, and SNPs therein, are compared to a reference genome so as to provide SNP information. In step 907, based on at least the SNP information, timestamp, mutation rate and reference genome, a dated phylogenetic tree is generated. In step 908, the dated phylogenetic tree is updated based on updating rules, thereby generating an updated phylogenetic tree.

The embodiments disclosed provide a technical solution to a technical problem. One technical problem being solved is generating a model comprising temporal and interconnected relations and information on transmission dynamics for a species, for example, species of pathogenic microbes. Such problem may be evident during an outbreak, for example. For example, a hospital environment may become a feeding ground for pathogenic microbes, the progression and transmission of which should be understood. In practice, this is problematic because a phylogenetic tree does not typically provide information on temporal connections between samples or evolutionary progression of a species.

The embodiments herein overcome these limitations by using computational methods and apparatuses in order to obtain information on transmission dynamics, for example, during an outbreak, by creating a dated phylogenetic tree, which is then optionally updated. In this manner, ancestral nodes, parent nodes and child nodes as well as connections thereof, which would otherwise remain unknown or ambiguous, are provided. This results in several advantages. The processing provides a simplified updated dated phylogenetic tree construction, as a parent node and connections to child nodes thereof may be made redundant based on obtain genetic information and differences thereof. Other technical improvements may also flow from these embodiments, and other technical problems may be solved.

It is to be understood that the embodiments of the invention disclosed are not limited to the particular structures, process steps, or materials disclosed herein, but are extended to equivalents thereof as would be recognized by those ordinarily skilled in the relevant arts. It should also be understood that terminology employed herein is used for the purpose of describing particular embodiments only and is not intended to be limiting.

Reference throughout this specification to “one embodiment” or “an embodiment” means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. In addition, various embodiments and example of the present invention may be referred to herein along with alternatives for the various components thereof. It is understood that such embodiments, examples, and alternatives are not to be construed as de facto equivalents of one another, but are to be considered as separate and autonomous representations of the present invention.

Furthermore, the described features, structures, or characteristics may be combined in any suitable manner in one or more embodiments. In the following description, numerous specific details are provided, such as examples of lengths, widths, shapes, etc., to provide a thorough understanding of embodiments of the invention. One skilled in the relevant art will recognize, however, that the invention can be practiced without one or more of the specific details, or with other methods, components, materials, etc. In other instances, well-known structures, materials, or operations are not shown or described in detail to avoid obscuring aspects of the invention.

While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

The verbs “to comprise” and “to include” are used in this document as open limitations that neither exclude nor require the existence of also un-recited features. The features recited in depending claims are mutually freely combinable unless otherwise explicitly stated. Furthermore, it is to be understood that the use of “a” or “an”, i.e. a singular form, throughout this document does not exclude a plurality.

### INDUSTRIAL APPLICABILITY

At least some of the embodiments find industrial applicability in biotechnology, outbreak and transmission dynamics.

### Acronyms List

AMR antimicrobial resistance
ASIC application specific integrated circuit
DAG directed acyclic graph
DNA deoxyribonucleic acid
DSP digital signal processor
FPGA field-programmable gate array
MCU microcontroller unit
RAM random access memory
ROM read-only memory
SEIR Susceptible-Exposed-Infectious-Recovered model
SIR Susceptible-Infectious-Recovered model
SNP single nucleotide polymorphism
SNV single nucleotide variant
WAN wide area network

REFERENCE SIGNS LIST

| | |
|---|---|
| 104 | Timestamp |
| 108 | Mutation rate |
| 110 | SNP information |
| 200 | Computing device |
| 201 | Instructions (generation rules) |
| 202 | Instructions (updating rules) |
| 210 | Common ancestor |
| 240 | Phylogenetic tree |
| 250 | Dated phylogenetic tree |
| 251 | Simplified dated phylogenetic tree |
| 260 | Node |
| 262 | Sample node |
| 263 | Internal node |
| 264 | Non-sample node |
| 265 | Parent node |
| 266 | Child node |
| 270 | Phylogenetic tree |
| 280 | Edge |
| 281 | SNP evolutionary distance |
| 282 | Weight of edge |
| 300 | Timeline |
| 301-307 | Steps |
| 400 | Apparatus |
| 401 | Processor |
| 402 | Memory |
| 403 | Communication interface |
| 462A | Sample node |
| 464 | Non-sample node |
| 500 | Timeline |
| 564A-C | Internal nodes |
| 601 | Patient |
| 601A | Sample genomic data |
| 662A-B | Sample genomic data |
| 662R | (Reference) sample genomic data |
| 602 | Hospital staff |
| 603 | Hospital equipment |
| 604 | Surface |
| 700 | Apparatus |
| 801-806 | Steps |
| 901-907 | Steps |

1.-45. (canceled)

46. A method for generating a transmission timeline, the method comprising:
obtaining a reference genome for a species;
obtaining a timestamp for each sample of a plurality of samples, wherein at least one sample is from said species;
obtaining sample genomic data corresponding to each sample;
obtaining, based on a difference between the reference genome and each sample genomic data, SNP information from each sample in the plurality of samples;
obtaining a mutation rate for said species;
determining, based on the SNP information, SNP evolutionary distance from the reference genome for each sample;
determining, based on the SNP information, SNP evolutionary distance between each sample; and
generating a dated phylogenetic tree based on: the SNP information, the SNP evolutionary distances from the reference genome, the SNP evolutionary distance between the samples, the mutation rate, generation rules, and the corresponding timestamps,
said dated phylogenetic tree comprising sample nodes and non-sample nodes,
wherein each sample corresponds to a node.

47. The method according to claim 46, wherein the dated phylogenetic tree is updated, for example in an updating process based on updating rules, in order to obtain an updated phylogenetic tree, wherein the updated phylogenetic tree has a different topology from the dated phylogenetic tree.

48. The method according to claim 47, wherein the updated phylogenetic tree has fewer non-sample nodes than the dated phylogenetic tree.

49. The method according to claim 46, wherein the dated phylogenetic tree comprises edges between nodes, said edges having values corresponding to the node-to-node SNP evolutionary distance.

50. The method according to claim 46, wherein the SNP information comprises: the SNP position in the reference genome, original nucleotide of the reference genome, and the mutated nucleotide of the sample.

51. The method according to claim 46, wherein the obtaining of the SNP information is done using SNP calling.

52. The method according to claim 46, wherein the generation rules comprise:
wherein if a first child node has a null SNP evolutionary distance to an original parent node of said first child node:
promoting said first child node to a promoted parent node, said promoted parent node comprising the SNP evolutionary distance of the original parent node; and
attaching other child nodes of the original parent node to the promoted parent node.

53. The method according to claim 52, wherein the generation rules further comprise:
wherein if the timestamp with respect to SNP evolutionary distance between the first child node and the original parent node is less than the mutation rate of the species, for example a typical mutation rate of the species, excluding the promotion of the first child node.

54. The method according to claim 46, wherein some nodes are internal nodes which correspond to a common ancestor of at least two child nodes.

55. The method according to claim 46, wherein the species is at least one of Acinetobacter baumannii, Enterococcus faecium, Candida auris, Escherichia coli or Staphylococcus aureus.

56. The method according to claim 46, wherein the species is Escherichia coli.

57. The method according to claim 46, wherein the obtaining of the sample genomic data comprises:
receiving sequence reads of the sample from the species; and
assembling said sequence reads thereby obtaining sample genomic data.

58. The method according to claim 46, wherein the obtaining of the sample genomic data comprises:
obtaining at least one sample from a sample source, wherein the sample source is, for example a patient, a healthcare worker, a medical device, an implement, or an environmental sample, wherein said sample comprises or is suspected to comprise at least one a pathogenic microbe;
preparing a pure culture from said sample;
isolating genomic DNA from said pure culture; and
sequencing the isolated genomic DNA to obtain sequence reads.

59. The method according to claim 46, wherein said plurality of samples comprises both patient and environmental samples.

60. An apparatus comprising at least one processing core, at least one memory including computer program code, the at least one memory and the computer program code being configured to, with the at least one processing core, cause the apparatus at least to:
obtain a reference genome for a species;
obtain a timestamp for each sample of a plurality of samples, wherein at least one sample is from said species;
obtain sample genomic data corresponding to each sample;
obtain, based on a difference between the reference genome and each sample genomic data, SNP information from each sample in the plurality of samples;
obtain a mutation rate for said species;
determine, based on the SNP information, SNP evolutionary distance from the reference genome for each sample;
determine, based on the SNP information, SNP evolutionary distance between each sample; and
generate a dated phylogenetic tree based on: the SNP information, the SNP evolutionary distances from the reference genome, the SNP distance between the samples, the mutation rate, generation rules and the corresponding timestamps, a dated phylogenetic tree, said tree comprising sample nodes and non-sample nodes, wherein each sample corresponds to a node.

61. The apparatus of claim 60, wherein the apparatus obtains the sample genomic data from a wide area network.

62. The apparatus according to claim 60, wherein the dated phylogenetic tree is updated based on updating rules in order to obtain an updated phylogenetic tree, wherein the updated phylogenetic tree has a different topology from the dated phylogenetic tree.

63. The apparatus according to claim 62, wherein the updated phylogenetic tree has fewer non-sample nodes than the dated phylogenetic tree.

64. The apparatus according to claim 60, wherein the dated phylogenetic tree comprises edges between nodes, said edges having values corresponding to the node-to-node SNP evolutionary distance.

65. A non-transitory computer readable medium having stored thereon a set of computer readable instructions that, when executed by at least one processor, cause an apparatus to at least:
obtain a reference genome for a species;
obtain a timestamp for each sample of a plurality of samples, wherein at least one sample is from said species;
obtain sample genomic data corresponding to each sample;
obtain, based on a difference between the reference genome and each sample genomic data, SNP information from each sample in the plurality of samples;
obtain a mutation rate for said species;
determine, based on the SNP information, SNP evolutionary distance from the reference genome for each sample;
determine, based on the SNP information, SNP evolutionary distance between each sample; and
generate a dated phylogenetic tree based on the SNP information, the SNP evolutionary distances from the reference genome, the SNP distance between the samples, the mutation rate, generation rules and the corresponding timestamps, a dated phylogenetic tree, said tree comprising sample nodes and non-sample nodes, and wherein each sample corresponds to a node.
